(19) United States
(12) Patent Application Publication
(10) Pub. No.: US 2010/0317626 A1
(43) Pub. Date: Dec. 16, 2010

(54) The Use Of (3-Amino-2-Fluoropropyl) Phosphinic Acid For Treatment Of NERD

(75) Inventors: Goran Hasselgren, Aadorf (CH); Anders Lehmann, Molndal (SE); Hans Rydholm, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 12/669,845

(22) PCT Filed: Jul. 24, 2008
(86) PCT No.: PCT/SE2008/050890

§ 371 Date: Jul. 15, 2010

Related U.S. Application Data
(60) Provisional application No. 60/951,744, filed on Jul. 25, 2007.

(51) Int. Cl. A61K 31/66; A61P 1/04

(52) U.S. Cl. 514/114

(57) ABSTRACT

The present invention is directed to the use of (3-Amino-2-fluoropropyl)phosphinic acid or an optical isomer thereof, or a pharmaceutically acceptable salt thereof, for the treatment or prevention of non-erosive reflux disease (NERD).

### FIELD OF THE INVENTION

The present invention is directed to the use of (3-Amino-2-fluoropropyl)phosphinic acid or an optical isomer thereof, or a pharmaceutically acceptable salt thereof, for the treatment or prevention of non-erosive reflux disease (NERD).

### BACKGROUND OF THE INVENTION

The lower esophageal sphincter (LES) is prone to relaxing intermittently, known as transient lower esophageal sphincter relaxation (TLESR). As a consequence, fluid from the stomach can pass into the esophagus since the mechanical barrier is temporarily lost at such times, an event hereinafter referred to as “reflux”. Gastroesophageal reflux disease (GERD) is one of the most common upper GI disorders with a prevalence between 10-20% in the Western World. GERD patients are subdivided into two main categories: Erosive reflux disease (ERD) and non-erosive reflux disease (NERD). While the latter frequently is viewed as a milder form, the severity and incidence of symptoms are identical in the two groups. The distribution of patients into the two groups differs between studies but in general, NERD comprise at least half of the GERD population (Martinez S D, Malagon I R, Garewal H S, Cui H, Fass R. Alimentary Pharmacology & Therapeutics 2003; 17:537-45).

A new definition of gastro-esophageal reflux disease (GERD), has been disclosed by Vakil N et al. in Am J Gastroenterol 2006; 101: 1900-1920; “The Montreal Definition and Classification of Gastroesophageal Reflux Disease: A Global Evidence Based Consensus”).

Non-erosive reflux disease (NERD) is a reflux disorder that does not lead to esophagitis. Patients suffering from NERD have normal esophageal mucosa when judged by ordinary, normal resolution, endoscopy and are typically classified as having endoscopy-negative reflux disease. NERD patients may have abnormal or normal acid exposure, i.e. the patients may suffer from weakly acidic or alkaline reflux.

Dekel R., Fass R., Minerva Gastroenterol. Dietol. (2003), 49(4), 277-287 report the use of baclofen to reduce the rate of transient lower esophageal sphincter relaxations and thereby treatment of NERD.

Koek G. H. et al., Gut (2003), 52, 1397-1402 report the use of baclofen in patients with persistent non-acid duodenogastroesophageal reflux. Baclofen was shown to inhibit the occurrence of bile reflux and reflux symptoms.

Baclofen is however associated with side effects such as drowsiness, vertigo, psychiatric disturbances, insomnia, slurred speech, ataxia, hypotonia, hypotension, fatigue, confusion, headache, rash, nausea, constipation and polyuria. The CNS side effect profile of baclofen thus limits the utility of the compound in the treatment of NERD.

### OUTLINE OF THE INVENTION

An object of the present invention was to find a new method of treating non-erosive reflux disease (NERD), with less side effects than those associated with the use of baclofen.

An aspect of the present invention is (3-Amino-2-fluoropropyl)phosphinic acid for the treatment of non-erosive reflux disease (NERD).

An aspect of the present invention is directed to the use of (3-Amino-2-fluoropropyl)phosphinic acid for the manufacture of a medicament for the treatment or prevention of NERD.

A further aspect of the present invention is the use of (2R)-(3-Amino-2-fluoropropyl)phosphinic acid for the manufacture of a medicament for the treatment or prevention of NERD.

Still a further aspect of the invention is (2R)-(3-Amino-2-fluoropropyl)-phosphinic acid for the treatment or prevention of NERD.

A further aspect of the present invention is a method for the treatment and/or prevention of NERD, wherein a pharmaceutically and pharmacologically effective amount of (3-Amino-2-fluoropropyl)phosphinic acid is administered to a subject in need of such prevention or treatment.

A further aspect of the present invention is a method for the treatment and/or prevention of NERD, wherein a pharmaceutically and pharmacologically effective amount of (2R)-(3-Amino-2-fluoropropyl)phosphinic acid is administered to a subject in need of such prevention or treatment.

Yet a further aspect of the invention is (2S)-(3-Amino-2-fluoropropyl)phosphinic acid for the treatment of non-erosive reflux disease (NERD).

A further aspect of the invention is directed to the use of (2S)-3-Amino-2-fluoropropyl)phosphinic acid for the manufacture of a medicament for the treatment or prevention of NERD.

A further aspect of the present invention is a method for the treatment and/or prevention of NERD, wherein a pharmaceutically and pharmacologically effective amount of (2S)-(3-Amino-2-fluoropropyl)phosphinic acid is administered to a subject in need of such prevention or treatment.

Compounds useful in accordance with the present invention are of amphoteric nature and may be presented in the form of internal salts. Compounds used herein can also form acid addition salts and salts with bases. Such salts are pharmaceutically acceptable acid addition salts, as well as pharmaceutically acceptable salts formed with bases. Examples of acids useful for the formation of such salts include, for example, mineral acids such as hydrochloric, hydrobromic, sulfuric, or phosphoric acid or organic acids such as sulfonic acids and carboxylic acids. Examples of bases useful for the formation of salts are, for example, alkali metal salts, e.g. sodium or potassium salts, or alkaline earth metal salts, e.g. calcium or magnesium salts, as well as ammonium salts, such as those with ammonia or organic amines.

A compound useful in the present invention can be in the form of a racemate or in the form of the single enantiomers.

Compounds useful in accordance with the present invention may also be present in the form of solvates, e.g. hydrates, or different crystal forms when used as described herein.

Non-erosive reflux disease (NERD) is a reflux disorder that does not lead to esophagitis. Patients suffering from NERD have microscopically normal esophageal mucosa when judged by ordinary, normal resolution, endoscopy and are typically classified as having endoscopy-negative reflux disease. NERD patients may have abnormal or normal acid exposure, i.e. the patients may suffer from weakly acidic or alkaline reflux.

The wording “treatment” as used herein also includes prevention or prophylaxis, unless there are specific indications to the contrary.

Compounds useful in accordance with the present invention can be prepared as described in WO01/42252.

### PHARMACEUTICAL FORMULATIONS

For clinical use, the active compound as used in accordance with the present invention may be formulated into a pharmaceutical formulation for oral administration. Also, parenteral or any other route of administration may be contemplated to the skilled man in the art of formulations. Thus, the active compound as used in accordance with the invention may be formulated with at least one pharmaceutically and pharmacologically acceptable carrier or adjuvant. The carrier may be in the form of a solid, semi-solid or liquid diluent.

In the preparation of an oral pharmaceutical formulation of a compound useful in accordance with the invention, the active compound to be formulated may be mixed with solid powdered ingredients, fillers, disintegrating agents and lubricating agents. The mixture is then processed into granules and/or compressed into tablets.

Soft gelatine capsules may be prepared with a capsule containing a mixture of the active compound and other suitable pharmaceutical agents and/or vehicles for soft gelatine capsules. Hard gelatine capsules may contain the active compound together with solid powdered ingredients.

Liquid preparations for oral administration may be prepared in the form of syrups or suspensions, e.g. solutions or suspensions, containing the active compound and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of the active compound in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

A daily dose of (3-Amino-2-fluoropropyl)phosphinic acid, (2R)-(3-Amino-2-fluoropropyl)phosphinic acid, or (2S)-(3-Amino-2-fluoropropyl)phosphinic acid or a salt of any one of said compounds useful in accordance with the invention, may be up to 2200 mg per day, such as up to 480 mg per day. The compound may for example be administered once or twice daily. In one embodiment of the invention the compound may be administered in a dose of 240 mg bid (i.e. twice daily amounting to 480 mg per day).

In yet an embodiment, the daily dose of a compound as used in accordance with the invention may be administered in a dosage such as 30 mg bid, 60 mg bid, 120 mg bid and 240 mg bid. The wording bid means that the compound is administered twice daily, and thus the daily dose of the compound may be 60 mg, 120 mg, 240 mg and 480 mg.

The wording “compound” or “active compound” as used in the specification and patent claims is herein defined as (3-Amino-2-fluoropropyl)phosphinic acid, (2R)-(3-Amino-2-fluoropropyl)phosphinic acid, (2S)-(3-Amino-2-fluoropropyl)phosphinic acid, or a pharmaceutically and pharmacologically acceptable salt of any one of said compounds, as well as a crystalline form of any one of said compounds, or a crystalline form of a salt thereof.

Also within the scope of the invention is the use of a crystalline form of any one of (3-Amino-2-fluoropropyl)phosphinic acid or a pharmaceutically acceptable salt thereof; (2R)-(3-Amino-2-fluoropropyl)phosphinic acid or a pharmaceutically acceptable salt thereof; or (2S)-(3-amino-2-fluoropropyl)phosphinic acid or a pharmaceutically acceptable salt thereof.

(2R)-(3-Amino-2-fluoropropyl)phosphinic acid may exist in different crystal forms such as Form A and Form B.

Preparation of (2R)-(3-Amino-2-fluoropropyl)phosphinic Acid, Form A

320 g (1.11 moles) (2R)-3-[(tert-butoxycarbonyl)amino]-2-fluoro-propyl phosphinic acid ammonium salt dissolved in methanol (960 ml, 23.72 moles) was treated with sulphuric acid (105.43 ml, 1.90 moles) at 55° C. After complete reaction, the reaction mixture was cooled to 30° C. and pH was adjusted to approximately 5 by addition of ammonium acetate dissolved in methanol (180 g, 2.34 moles, 420 ml methanol). During the pH-adjustment ammonium sulphate remaining ammonium acetate and other salts precipitated. The neutralised reaction mixture was clear filtrated. Isopropanol (3.84 L, 50.23 moles) was to added at 50° C. and (2R)-(3-Amino-2-fluoropropyl)phosphinic acid, Form A, crystallised. The slurry was cooled to 0° C. The crystals were isolated and dried under vacuum.

¹H-NMR (400 MHz, D₂O): δ1.93 (1H, m), 2.13 (1H, m), 3.31 (2H, m), 5.14 (1H, dm, J=50 Hz), 7.07 (1H, d, J=528 Hz).

The crystals were analysed by X-ray powder diffraction (XRPD). The diffractogram of form A shows the following d-values given in Ångström and relative intensities:

Form A

| | Relative | | Relative |
|---|---|---|---|
| d-value (Å) | Intensity | d-value (Å) | Intensity |
| 7.8 | vs | 3.08 | w |
| 7.6 | w | 2.96 | vw |
| 5.8 | m | 2.92 | m |
| 4.44 | vw | 2.80 | m |
| 4.34 | w | 2.69 | w |
| 4.18 | s | 2.59 | w |
| 4.04 | s | 2.57 | w |
| 3.91 | s | 2.53 | s |
| 3.88 | vs | 2.48 | w |
| 3.79 | m | 2.46 | w |
| 3.58 | w | 2.41 | vw |
| 3.54 | w | 2.33 | vw |
| 3.43 | vw | 2.28 | vw |
| 3.32 | m | | |

The relative intensities are presented by the following definitions.

| Definitions used | % Relative Intensity |
|---|---|
| vs (very strong): | 100-70 |
| S (strong): | 70-40 |
| m (medium): | 40-10 |
| w (weak): | 10-5 |
| vw (very weak): | <5 |

The relative intensities were derived from diffractograms measured with variable slits.

Preparation of (2R)-(3-Amino-2-fluoropropyl)phosphinic Acid, Form B

40 g of (2R)-(3-Amino-2-fluoropropyl)phosphinic acid form A, was added to 150 mL methanol and 65 mL water. The slurry was heated to 40° C. until all was dissolved. 320 mL of acetone was added to the solution over 10 hrs. The slurry was stirred at 40° C. for 33 hours. Then obtained crystals were filtered and dried in vacuum at 40° C. overnight. 36.67 g of (2R)-(3-Amino-2-fluoropropyl)phosphinic acid, form B, was obtained after drying.

¹H-NMR (400 MHz, D₂O): δ1.95 (1H, m), 2.15 (1H, m), 3.33 (2H, m), 5.16 (1H, dm, J=50 Hz), 7.08 (1H, d, J=528 Hz).

The crystals were analysed by X-ray powder diffraction (XRPD). The diffractogram of form B shows the following d-values given in Ångström and relative intensities:

Form B

| | Relative | | |
|---|---|---|---|
| d-value (Å) | Intensity | d-value (Å) | Relative Intensity |
| 7.7 | vw | 3.09 | vw |
| 6.2 | s | 3.00 | vw |
| 6.0 | w | 2.96 | w |
| 5.6 | w | 2.93 | w |
| 4.83 | vw | 2.87 | s |
| 4.75 | vw | 2.82 | w |
| 4.10 | s | 2.76 | w |
| 4.05 | vs | 2.69 | w |
| 3.88 | m | 2.60 | s |
| 3.73 | vw | 2.47 | s |
| 3.63 | w | 2.35 | w |
| 3.59 | m | 2.28 | w |
| 3.36 | m | 2.25 | s |
| 3.28 | m | | |

The relative intensities are presented by the following definitions:

| Definitions used | % Relative Intensity |
|---|---|
| vs (very strong): | 31-100 |
| s (strong): | 8.1-31 |
| m (medium): | 3.1-8.1 |
| w (weak): | 0.7-3.1 |
| vw (very weak): | 0-0.7 |

The relative intensities are derived from diffractograms measured with variable slits.

### Biological Evaluation

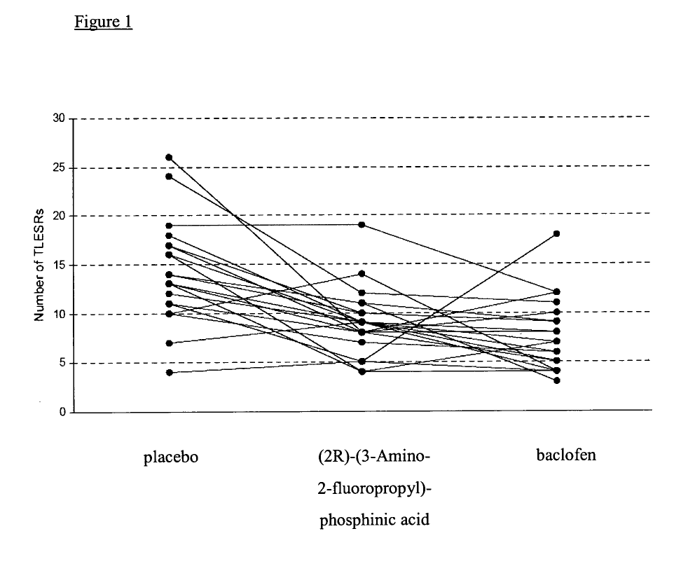

A clinical study of (2R)-(3-Amino-2-fluoropropyl)phosphinic acid was performed. The study was a single-blind, randomised, parallel group, placebo-controlled study in healthy volunteers. Each subject was given 2 doses of (2R)-(3-Amino-2-fluoropropyl)phosphinic acid in escalating order and one placebo dose. A dose of (2R)-(3-Amino-2-fluoropropyl)phosphinic acid 0.8 mg/kg was compared to placebo and baclofen (40 mg) was used as a positive control. The order of treatments was randomised. The number of transient lower esophageal sphincter relaxations (TLESRs) 0-3 hours after intake of a standardised meal, (completed 1 hour after drug intake) were reduced by 36% and 47% for (2R)-(3-Amino-2-fluoropropyl)phosphinic acid and baclofen respectively, as compared to placebo (FIG. 1).

(2R)-(3-Amino-2-fluoropropyl)phosphinic acid showed to be efficacious for the inhibition of TLESRs, as well as being well tolerated.

1-5. (canceled)

6. A method for the treatment or prevention of non-erosive reflux disease comprising administering to a subject in need of such treatment or prevention a pharmaceutically and pharmacologically effective amount of (3-Amino-2-fluoropropyl)phosphinic acid or an optical isomer thereof, or a pharmaceutically acceptable salt thereof.

7. A method for the treatment or prevention of non-erosive reflux disease comprising administering to a subject in need of such treatment or prevention a pharmaceutically and pharmacologically effective amount of (2R)-(3-Amino-2-fluoropropyl)phosphinic acid or a pharmaceutically acceptable salt thereof.

8. A method according to claim 6, wherein the daily dose of the active compound is up to 2200 mg per day.

9. A method according to claim 7 wherein the daily dose of the active compound is up to 2200 mg per day.
